(19) United States
(12) Patent Application Publication
(10) Pub. No.: US 2004/0253596 A1
(43) Pub. Date: Dec. 16, 2004

(54) Bioanalytical recognition surface with optimized recognition element density

(76) Inventors: Michael Pawlak, Laufenburg (DE); Gert Ludwig Duveneck, Bad Krozingen (DE); Ning-Ping Huang, Basel (CH); Susan Margaret De Paul, Zurich (CH); Marcus Textor, Schaffhausen (CH); Rolf Hofer, Dachsen (CH); Eveline Schurmann-Mader, Zeihen (CH)

Correspondence Address: WENDEROTH, LIND & PONACK, L.L.P., 2033 K STREET N. W., SUITE 800, WASHINGTON, DC, 20006-1021, US

(21) Appl. No.: 10/487,720

(22) PCT Filed: Aug. 24, 2002
(86) PCT No.: PCT/EP02/09489

(30) Foreign Application Priority Data
Aug. 27, 2001 (CH) 1585/01

(51) Int. Cl. C12Q 1/68; C12M 1/34

(52) U.S. Cl. 435/6; 435/287.2

(57) ABSTRACT

The invention relates to a recognition surface on a carrier with an (in relation to the surface) optimal binding capacity for recognizing and binding one or more analytes from one or more samples brought into contact with this surface, wherein

a) said recognition surface comprises a mixture of specific biological or biochemical or synthetic recognition elements for the recognition and binding of said analytes with components which are “neutral” in respect of these analytes, i.e. which do not bind these analytes, and

b) said specific recognition elements, in relation to the entire recognition surface or any sub-surface thereof, take up less than a full monolayer.

The invention also relates to a method for the qualitative and/or quantitative detection of one or more analytes in one or more samples, wherein said samples and if necessary further reagents are brought into contact with a recognition surface according to the invention, and changes in optical or electronic signals resulting from the binding of the analyte or of further tracer substances used for the analyte detection are measured.

The invention relates to a recognition surface on a carrier with an (in relation to the surface) optimal binding capacity for recognizing and binding one or more analytes from one or more samples brought into contact with this surface, wherein

a) said recognition surface comprises a mixture of specific biological or biochemical or synthetic recognition elements for the recognition and binding of said analytes with components which are “neutral” in respect of these analytes, i.e. which do not bind these analytes, and

b) said specific recognition elements, in relation to the entire recognition surface or any sub-surface thereof, take up less than a full monolayer.

The invention also relates to a method for the qualitative and/or quantitative detection of one or more analytes in one or more samples, wherein said samples and if necessary further reagents are brought into contact with a recognition surface according to the invention, and changes in optical or electronic signals resulting from the binding of the analyte or of further tracer substances used for the analyte detection are measured.

For the detection of one or more analytes from a sample with a complex mixture of numerous substances there are widespread methods in which one or more so-called recognition elements which are of biological, biochemical or synthetic character are immobilized on a solid carrier before they are then brought into contact in immobilized form with said sample and the analytes contained therein bind to the recognition elements specific for them. In this case, the solid carrier may be both of macroscopic nature with a surface of square millimeters to square centimeters and also of microscopic nature, for example in the form of so-called beads, i.e. approximately spherical particles with typical diameters in the micrometer range. The surface of such a solid carrier with recognition elements immobilized thereon shall hereinafter be called a “recognition surface”.

Compared with methods in which the analytes and their recognition elements are brought together as reaction or binding partners in homogeneous liquid solution, these methods which are based on a solid carrier offer numerous advantages, for example an easier separation or differentiation of bound analyte molecules from the remainder of the sample. These advantages are gained with a restriction of the diffusive mixture between analyte molecules and recognition elements.

For the preparation of recognition surfaces for the highly efficient and highly selective binding of the one or more analytes to be detected in a sample, the quality of these surfaces is of major importance. To achieve the lowest possible limits of detection, it is desirable to immobilize in a small space as many recognition elements as possible in such a way that as many analyte molecules of one variety as possible may then be bound in the later detection process. At the same time it is desirable on immobilization to maintain as high a degree of reactivity and biological or biochemical functionality of the recognition elements as possible, i.e. to minimize any signs of denaturation resulting from the immobilization. A further objective is as far as possible to prevent the nonspecific binding or adsorption of analyte molecules which in many cases have the effect of restricting the limits of detection attainable.

In WO 84/01031 and in US Pat. Nos. 5,807,755, 5,837,551 and 5,432,099, immobilization of specific recognition elements for the analyte is proposed in the form of small “spots” with surface areas in some cases markedly less than 1 mm² on solid carriers. It is postulated as an advantage in this respect that, through the binding of only a small part of the analyte molecules present, the concentration of the analyte may be measured in a manner which is only dependent on the incubation time, but—in the absence of a continuous flow—is essentially independent of the absolute sample volume. At the same time, the density of bound analyte molecules achieved in these spots can be expected to be higher with such an arrangement than if they were distributed over an area completely covered with recognition elements.

Here too, however, as in the case of a large-area immobilization of recognition elements on a macroscopic surface, a high density of immobilized recognition elements in the measurement areas thereby created can have the effect of limiting the maximum number of analyte molecules that can be bound to the surface. A major reason for such a limitation of binding capacity may be steric hindrance.

The present invention resolves the problem of providing a recognition surface with a maximum binding capacity and at the same time minimizes the extent of unwanted nonspecific binding to the surface. The mixture of specific recognition elements and components which are “neutral” to the analyte, i.e. which do not bind these analytes, furthermore has the effect of enhancing the binding capacity of said specific recognition elements by preventing or hampering a denaturation of said recognition elements on the surface of the carrier which would otherwise compromise or even abolish the binding capacity. This mixture also has the effect of promoting a uniform distribution of the recognition elements within the recognition surface by preventing, for example, an accumulation of recognition elements in clusters after application in liquid solution and evaporation of the solution.

The first subject of the invention is a recognition surface with an (in relation to the surface) optimal binding capacity for recognizing and binding one or more analytes from one or more samples brought into contact with this surface, wherein

a) said recognition surface comprises a mixture of specific biological or biochemical or synthetic recognition elements for the recognition and binding of said analytes with components which are “neutral” in respect of these analytes, i.e. which do not bind these analytes, and

b) said specific recognition elements, in relation to the entire recognition surface or any sub-surface thereof, take up less than a full monolayer.

Said specific recognition elements, in relation to the entire recognition surface or any sub-surface thereof, preferably form one-tenth to half of a complete monolayer. It is also preferred if said specific recognition elements and components “neutral” to the analytes, in relation to the entire recognition surface or any sub-surface thereof, together form at least two-thirds of a complete monolayer.

For numerous applications, there is a wish to determine not only a single analyte, but multiple analytes simultaneously. A second subject of the invention is accordingly a structured recognition surface with an (in relation to the surface) optimal binding capacity for recognizing and binding one or more analytes from one or more samples brought into contact with this surface, wherein

a) said recognition surface in discrete, laterally separated measurement areas comprises a mixture of specific biological or biochemical or synthetic recognition elements for the recognition and binding of said analytes with components which are “neutral” in respect of these analytes, i.e. which do riot bind these analytes, and

b) said specific recognition elements, in relation to the surface area of the discrete measurement areas, take up less than a full monolayer.

It is also preferred for this variant if said specific recognition elements, in relation to the entire recognition surface or any sub-surface thereof, form one-tenth to half of a complete monolayer. It is also preferred for a structured recognition surface according to the invention if said specific recognition elements and components “neutral” to the analytes, in relation to the entire recognition surface or any sub-surface thereof, together form at least two-thirds of a complete monolayer.

Within the terms of the present invention, laterally separate measurement areas, as an integral part of a recognition surface according to the invention, should be defined by the surface area which encompasses the biological or biochemical or synthetic recognition elements immobilized thereon for the detection of an analyte from a liquid sample and the molecules which are “neutral” to said analytes and are mixed with the recognition elements. These areas may be present in any geometric form, for example in the form of points, circles, rectangles, triangles, ellipses or lines. It is possible that up to 1,000,000 measurement areas may be present in a two-dimensional arrangement, wherein a single measurement area may take up an area of 10⁻⁴ mm²-10 mm². The density of the measurement areas may typically amount to more than 10, preferably more than 100, especially preferably more than 1000 measurement areas per square centimeter.

Usually a recognition surface according to the invention is generated on a solid surface. There are numerous methods for applying the recognition elements to a carrier surface and various modes of interaction of these recognition elements with the element-bearing surface which ensure their immobilization. For example, the application and subsequent adhesion may take place by electrostatic interaction or more generally by physical adsorption. The orientation of the recognition elements is then generally statistical. There is also a risk that part of the recognition elements is washed away on addition of the sample containing the analyte and, if applicable, further reagents sequentially to the recognition surface. It may therefore be of advantage if an adhesion-promoting layer is applied to the carrier surface for the immobilization of biological or biochemical or synthetic recognition elements. For many applications, for example in the case of analyte detection based on optical methods, it is an advantage if this adhesion-promoting layer is transparent at least at an excitation wavelength. The thickness of such an optional adhesion-promoting layer is preferably less than 200 nm, but especially preferably less than 20 nm. The optional adhesion-promoting layer may for example comprise a chemical compound from the groups of silanes, functionalized silanes, epoxides, functionalized, charged or polar polymers and “self-assembled passive or functionalized monolayers or multilayers”.

It is preferred if discrete (laterally separated) measurement areas, as an integral part of this recognition surface, are generated by the laterally selective application of biological or biochemical or synthetic recognition elements on a surface of a carrier or on an adhesion-promoting layer additionally applied to a carrier surface, preferably using one or more methods from the group of methods comprising ink-jet spotting, mechanical spotting by means of pin, pen or capillary, micro-contact printing, fluidic contact of the measurement areas with the biological or biochemical or synthetic recognition elements through their application in parallel or intersecting microchannels, upon exposure to pressure differences or to electric or electromagnetic potentials, and photochemical or photolithographic immobilization methods.

It is further advantageous if regions between the laterally separated measurement areas are “passivated” in order to minimize nonspecific binding of analytes or their tracer compounds, i.e. if compounds are deposited between the laterally separated measurement areas which are “chemically neutral” to the analyte or one of its tracer compounds, formed preferably for example from groups comprising albumins, especially bovine serum albumin or human serum albumin, casein, nonspecific polyclonal or monoclonal, heterologous or empirically nonspecific antibodies for the analyte or analytes to be determined (especially for immunoassays), detergents (such as Tween 20®), fragmented natural or synthetic DNA not hybridizing with polynucleotides to be analyzed, such as extract from herring or salmon sperm (especially for polynucleotide hybridization assays), or also uncharged but hydrophilic polymers, such as polyethylene glycols or dextrans.

The biological or biochemical or synthetic recognition elements may be selected from the group comprising proteins, for example monoclonal or polyclonal antibodies and antibody fragments, peptides, enzymes, aptamers, synthetic peptide structures, glycopeptides, oligosaccharides, lectins, antigens for antibodies (e.g. biotin for streptavidin), proteins functionalized with additional binding sites (“tag proteins”, such as “histidine tag proteins”) and their complexing partners. Another group of compounds which are likewise preferred as recognition elements comprises nucleic acids (for example DNA, RNA, oligonucleotides) and nucleic acid analogs (e.g. PNA) and their derivatives with synthetic bases. A third preferred group of compounds as recognition elements comprises soluble, membrane-bound proteins and proteins isolated from a membrane, such as receptors and their ligands.

Also said “neutral” components which do not bind the analyte or analytes may be selected from groups comprising albumins, especially bovine serum albumin or human serum albumin, casein, nonspecific, polyclonal or monoclonal, heterologous or for the analyte or analytes to be determined empirically nonspecific antibodies (especially for immunoassays), detergents (such as Tween 20), fragmented natural or synthetic DNA not hybridizing with polynucleotides to be analyzed, such as a herring or salmon sperm extract (especially for polynucleotide hybridization assays), or also uncharged, but hydrophilic polymers, such as polyethylene glycols or dextrans.

In WO 00/65352, coatings with graft copolymers having a polyionic main chain, e.g. a chain (electrostatically) binding to a carrier, and “noninteractive” (adsorption-resistant) side chains, are described for the coating of bioanalytic sensor platforms or implants for medical applications.

A preferred embodiment of a recognition surface according to the invention comprises the recognition elements being bound to the free end or close to the free end of a wholly or partly functionalized, “noninteractive” (adsorption-resistant, uncharged) polymer, wherein said “noninteractive” (adsorption-resistant, uncharged) polymer as a side chain is bound to a charged, polyionic polymer as the main chain and, together with this polymer, forms a polyionic, multifunctional copolymer.

A large group of embodiments of a recognition element according to the invention in this case comprises the polyionic polymer main chain being cationically (positively) charged at approximately neutral pH. It may for example be selected from the group of polymers comprising amino acids with a positive charge at approximately neutral pH, polysaccharides, polyamines, polymers of quartemary amines and charged synthetic polymers. The cationic polymer main chain may also comprise one or more molecular groups from the group comprising lysine, histidine, arginine, chitosan, partially deacetylated chitin, amine-containing derivatives of neutral polysaccharides, polyaminostyrene, polyamine acrylates, polyamine methacrylates, polyethylene imines, polyamine ethylenes, polyaminostyrenes and N-alkyl derivatives thereof.

Further suitable molecular groups as an integral part of a polyionic main chain are described in WO 00/65352, which is introduced here in its entirety as an integral part of this description.

A characteristic of another large group of embodiments is that the polyionic polymer main chain is anionically (negatively) charged at approximately neutral pH. Within this group, the cationic main chain may be selected from the group of polymers comprising amino acids with associated groups having a negative charge at approximately neutral pH, polysaccharides and charged synthetic polymers with negatively charged groups.

The cationic polymer main chain may comprise one or more molecular groups from the group comprising polyasparaginic acid, polyglutamic acid, alginic acid or derivatives thereof, pectin, hyaluronic acid, heparin, heparin sulfate, chondroitin sulfate, dermatan sulfate, dextran sulfate, polymethyl methacrylic acid, oxidized cellulose, carboxymethylated cellulose, maleic acid and fumaric acid.

The “noninteractive” (uncharged) polymer” as side chain may be selected from the group comprising poly(alkylene glycols), poly(alkylene oxides), neutral water-soluble polysaccharides, polyvinyl alcohols, poly-N-vinyl pyrrolidones, phosphorylcholine derivatives, noncationic poly(meth)acrylates and combinations thereof.

The biological or biochemical or synthetic recognition elements are preferably bound to the free end or close to the free end of the “noninteractive” side chain via reactive groups: It is especially preferred if said reactive groups are selected from the group comprising hydroxy (—OH), carboxy (—COOH), esters (—COOR), thiols (—SH), N-hydroxysuccinimide, maleimidyl, quinone, vinylsulfone, nitrilo triacetic acid (NTA) and combinations thereof.

Numerous further suitable polymers and preferred embodiments are additionally described in WO 00/65352.

It is generally preferred if a recognition surface according to the invention is applied to an essentially optically transparent carrier.

The term “essentially optically transparent” is understood to mean that a layer thus characterized is a minimum of 95% transparent at least at the wavelength of light delivered from an external light source for its optical path perpendicular to said layer, provided the layer is not reflecting. In the case of partially reflecting layers, “essentially optically transparent” is understood to mean that the sum of transmitted and reflected light and, if applicable, light in-coupled into a layer and guided therein amounts to a minimum of 95% of the delivered light at the point of incidence of the delivered light.

Light delivered from an external light source in the direction of the recognition surface, i.e. both through a carrier in the direction of the recognition surface and also from the opposite side, if applicable through a medium located over the recognition surface, shall according to the present invention be defined generally as “excitation light”. This excitation light may for example serve for the excitation of luminescence or, more specifically, of fluorescence or phosphorescence.

The essentially optically transparent carrier preferably comprises a material from the group comprising moldable, sprayable or millable plastics, metals, metal oxides, silicates, such as glass, quartz or ceramics.

A possible embodiment comprises the recognition surface according to the invention being deposited on an adhesion-promoting layer which is applied to an essentially optically transparent carrier and which is likewise essentially optically transparent.

A special embodiment comprises recesses being formed in the surface of said carrier for the creation of sample compartments. These recesses typically have a depth of 20 μm to 500 μm, preferably 50 μm to 300 μm.

The essentially optically transparent carrier preferably comprises a continuous optical waveguide or an optical waveguide divided into individual waveguiding areas. It is especially advantageous if the optical waveguide is an optical film waveguide with a first essentially optically transparent layer (a) facing the recognition surface on a second essentially optically transparent layer (b) with a refractive index lower than that of layer (a).

It is preferred if said optical film waveguide is essentially planar. Planar optical film waveguides and modifications thereof which are suitable as carriers are described for example in patent applications WO 95/33197, WO 95/33198, WO 96/35940, WO 98/09156, WO 99/40415, PCT/EP 00/04869 and PCT/EP 01/00605. The content of these patent applications is therefore introduced in its entirety as an integral part of this description.

It is preferred if, for the in-coupling of excitation light into the optically transparent layer (a), this layer is in optical contact with one or more optical in-coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

It is advantageous if the excitation light is in-coupled into the optically transparent layer (a) using one or more grating structures (c) which are featured in the optically transparent layer (a).

A characteristic of a further variant is that the light guided in the optically transparent layer (a) is out-coupled using one or more grating structures (c′) which are featured in the optically transparent layer (a) and have the same or different period and grating depth as grating structures (c).

A further subject of the invention is a method for the qualitative and/or quantitative detection of one or more analytes in one or more samples, wherein said samples and if necessary further reagents are brought into contact with a recognition surface according to the invention as described in one of the aforementioned embodiments, and changes in optical or electronic signals resulting from the binding of the analyte or of further tracer substances used for the analyte detection are measured.

A subject of the invention is also a method for the qualitative and/or quantitative detection of one or more analytes in one or more samples, wherein said samples and if necessary further reagents are brought into contact with a structured recognition surface according to the invention as described in one of the aforementioned embodiments, and changes in optical or electronic signals emanating from the discrete measurement areas as a result of the binding of the analyte or of further tracer substances used for the analyte detection are measured in a locally resolved manner.

In this case, one or more samples are preferably incubated beforehand with a mixture of the various tracer reagents for determining the analytes to be detected in said samples and these mixtures then brought into contact in a single addition step with a recognition surface according to the invention.

It is also preferred if the detection of one or more analytes is based on the determination of the alteration in one or more luminescences.

The excitation light from one or more light sources may be delivered in an epi-illumination arrangement. The excitation light may also be delivered in a transillumination arrangement.

A method is preferred which comprises the recognition surface, if necessary mediated via an adhesion-promoting layer, being arranged on an optical waveguide which is preferably essentially planar, the one or more samples with one or more analytes to be detected therein and, if necessary, further tracer reagents being brought, sequentially or in a single addition step after mixing with said samples, into contact with said recognition surface, and the excitation light from one or more light sources being in-coupled into the optical waveguide, as described hereinbefore for the optical film waveguide.

A characteristic of a special embodiment of the method according to the invention is that the detection of one or more analytes on a recognition surface takes place via a grating structure (c) or (c′) formed in the layer (a) of an optical film waveguide based on changes in the resonance conditions for the in-coupling of excitation light into layer (a) of a carrier formed as film waveguide or for out-coupling of light guided in layer (a), these changes resulting from binding of the analyte and/or further tracer reagents to their immobilized biological or biochemical or synthetic recognition elements.

Especially preferred is a variant of the method according to the invention wherein said optical waveguide is designed as an optical film waveguide with a first optically transparent layer (a) on a second optically transparent layer (b) with lower refractive index than that of layer (a), wherein excitation light is further in-coupled into the optically transparent layer (a) with the aid of one or more grating structures, which are featured in the optically transparent layer (a), and delivered to the measurement areas (d) located thereon as a guided wave, and wherein the luminescence of molecules capable of luminescence, generated in the evanescent field of said guided wave, is further determined using one or more detectors, and the concentration of one or more analytes is determined from the intensity of these luminescence signals.

In this case (1) the isotropically emitted luminescence or (2) luminescence in-coupled into the optically transparent layer (a) and out-coupled via grating structure (c) or (c′) or luminescences of both (1) and (2) may be measured simultaneously.

An integral part of the method according to the invention is that for the generation of luminescence, a luminescence dye or luminescent nanoparticle is used as a luminescence label, which can be excited and emits at a wavelength between 300 nm and 1100 nm.

The luminescence label is preferably bound to the analyte or, in a competitive assay, to an analog of the analyte or, in a multistep assay, to one of the binding partners of the immobilized biological or biochemical or synthetic recognition elements or to the biological or biochemical or synthetic recognition elements.

Another embodiment of the method comprises the use of a second luminescence label or further luminescence labels with excitation wavelengths either the same as or different from that of the first luminescence label and the same or different emission wavelength.

It is preferred here if the second luminescence label or further luminescence labels can be excited at the same wavelength as the first luminescence dye, but emit at different wavelengths.

In particular it is advantageous if the excitation spectra and emission spectra of the luminescence dyes used overlap only a little, if at all.

A variant of the method comprises using charge or optical energy transfer from a first luminescence dye serving as donor to a second luminescence dye serving as acceptor for the purpose of detecting the analyte.

Another embodiment of the method comprises determining changes in the effective refractive index on the measurement areas in addition to measuring one or more luminescences.

A further embodiment of the method comprises one or more luminescences and/or determinations of light signals at the excitation wavelength being performed using polarization selective procedures.

The one or more luminescences are preferably measured at a polarization that is different from the one of the excitation light.

An subject of the invention is a method according to one of the aforementioned embodiments for simultaneous or sequential, quantitative or qualitative determination of one or more analytes from the group of proteins, such as antibodies or antigens, receptors or ligands, chelators, functionalized proteins with one or more additional binding sites (“tag proteins” such as “histidine tag proteins”) and complexing partners thereof, oligonucleotides, DNA or RNA strands, DNA or RNA analogs, enzymes, enzyme cofactors or inhibitors, lectins and carbohydrates.

Possible embodiments of the process comprise the samples to be tested being, for example, in the form of naturally occurring body fluids, such as blood, serum, plasma, lymph or urine or tissue fluids or egg yolk.

Other embodiments comprise the sample to be tested being in the form of an optically turbid fluid, surface water, a soil or plant extract, or a biological or synthetic process broth.

It is also possible that the samples to be tested may be prepared from biological tissue parts or cell cultures.

A further subject of the invention is the use of a method according to the invention for quantitative or qualitative analyses for the determination of chemical, biochemical or biological analytes in screening methods in pharmaceutical research, combinatorial chemistry, clinical and pre-clinical development, for real-time binding studies and the determination of kinetic parameters in affinity screening and in research, for qualitative and quantitative analyte determinations, especially for DNA and RNA analytics and for the determination of genomic or proteomic differences in the genome, such as single nucleotide polymorphisms, for the measurement of protein-DNA interactions, for the determination of control mechanisms for mRNA expression and for protein (bio)synthesis, for the generation of toxicity studies and the determination of expression profiles, especially for the determination of biological and chemical marker compounds, such as mRNA, proteins, peptides or small-molecular organic (messenger) compounds, and for the determination of antibodies, antigens, pathogens or bacteria in pharmaceutical product research and development, human and veterinary diagnostics, agrochemical product research and development, for symptomatic and pre-symptomatic plant diagnostics, for patient stratification in pharmaceutical product development and for therapeutic drug selection, for the determination of pathogens, nocuous agents and germs, especially of salmonella, prions, viruses and bacteria, especially in food and environmental analytics.

### EXAMPLE

1. Materials

Poly(L-lysine)hydrobromide (molecular weight about 20 kDa), streptavidin from Streptomyces avidinii (molecular weight about 60 kDa), avidin from albumen (molecular weight about 66 kDa), biotinylated (i.e. bound to biotin) goat anti-rabbit immunoglobulin (anti-R-IgG biotin, molecular weight about 150 kDa) and biotinylated bovine serum albumin (BSA biotin, molecular weight about 66 kDa) were obtained from Sigma-Aldrich (Buchs, Switzerland). The N-hydroxysuccinimidyl ester of methoxypoly(ethyleneglycol)propionic acid (MeO-PEG-SPA, molecular weight 2 kDa) and the α-biotin-ω-N-hydroxysuccinimidyl ester of poly(ethyleneglycol)carbonate (biotin-PEG-CO₂-NHS, molecular weight 3.4 kDa) were obtained from Shearwater Polymers Inc. (Huntsville, USA). Rabbit immunoglobulin (anti-human albumin) (R-IgG, molecular weight about 150 kDa) and rabbit anti-bovine serum albumin (anti-BSA, molecular weight about 150 kDa) were obtained from DAKO (Glostrup, Denmark). All said antibody reagents were polyclonal. Control serum N (human) was obtained from Hoffmann-La Roche (Basel, Switzerland). 4-(2-hydroxyethyl)piperazine-1-ethanesulfonic acid (HEPES) and other chemicals for the preparation of buffers were obtained from Fluka (Buchs, Switzerland).

All aqueous solutions were prepared using ultrapure water (18 MΩcm) from an “EasyPure Reverse Osmosis System” (Barnstead Thermolyne, Dubuque, USA).

2. Carrier

A thin-film waveguide formed as a grating coupler (TiO₂—SiO₂-solgel as waveguiding layer on a glass substrate, coupling grating period in the waveguiding layer: 417 nm) (Mikrovakuum Ltd., Budapest, Hungary), with a 12 nm thick Nb₂O₅ layer deposited thereon by sputtering, serves as carrier. Before they were first used, these carriers, with Nb₂O₅ as uppermost layer, were sonicated for 10 minutes in 0.1 M HCl, thoroughly rinsed with ultrapure water, dry-blown with nitrogen and subsequently treated for 2 hours with oxygen plasma in a plasma cleaner/sterilizer PDC-32G (Harrick, Ossining, USA).

3. Synthesis of PLL-g-PEG and Derivatives Thereof

The synthesis of PLL-g-PEG has been described by Sawhney and Hubbell (A. S. Sawhney, J. A. Hubbell, Biomaterials 13 (1992) 863-870). The studies which serve as a basis for the present application used procedures based on a method developed by Elbert and Hubbell (D. L. Elbert, J. A. Hubbell, J. Biomed. Mater. Res. 42 (1998) 55-65). According to WO 00/65352, for PEG subchains with a molecular weight of 2 kDa, a grafting ratio between g=3 and g=5 has proved optimal for immobilization of a maximum possible quantity of polymers on negatively charged metal oxide surfaces with simultaneous assurance of minimal nonspecific binding (adsorption) of proteins to the metal oxide surfaces coated with these polymers. For the experiments described in this example, a ratio of g=3.5 is used. In this case, the percentage proportion of PEG chains with biotin bound thereto (“biotinylated PLL-g-PEG”) varies.

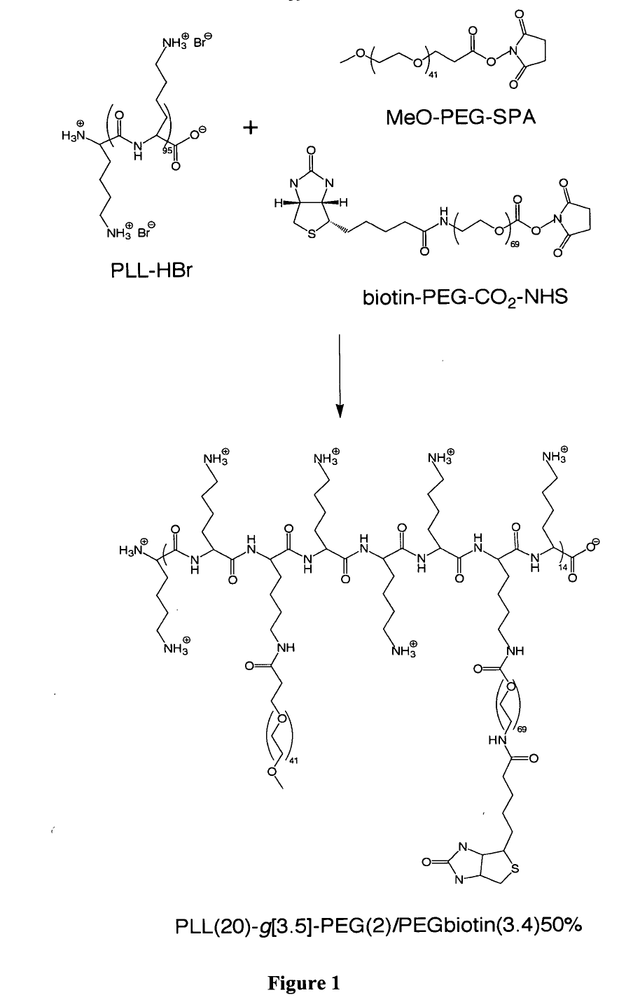

FIG. 1 shows a schematic representation of the synthesis of PLL-g-PEG. N-Hydroxysuccinimidyl esters both of biotinylated and of nonbiotinylated poly(ethyleneglycol) (“PEG”) are reacted with poly(L-lysine) (“PLL”) under stoichiometric conditions to manufacture the desired product. The details on this synthesis are described hereinafter under 3.1 and 3.2. For the biotinylated PEG chains, a chain length greater than that for methoxy-PEG is selected to ensure good accessibility of the polymer-bound biotin.

The nomenclature used hereinafter to describe the various PLL-g-PEG derivatives includes the molecular weights of the polymer subchains of the copolymers, the grafting ratio and the percentage of biotinylated PEGs. Accordingly, “PLL(20)-g[3.5]-PEG(2)/PEG biotin(3.4)30%” describes a polymer composed of a main chain of poly(L-lysine) with a molecular weight of 20 kDa and side chains of which 70% comprises poly(ethyleneglycol) with a molecular weight of 2 kDa and 30% biotinylated poly(ethyleneglycol) with a molecular weight of 3.4 kDa. The grafting ratio of 3.5 means that, on average, biotinylated or nonbiotinylated PEG chains in each case are bound to two of seven lysine groups (lysine units). Since all the polymers mentioned in this example were manufactured from identical precursor products, the abbreviation “PPB30” is also to be used as an alternative to “PLL-g-PEG/PEG biotin30%”. Corresponding abbreviations are used for other percentages of biotinylated PLL-g-PEGs.

3.1 Synthesis of PLL(20)-g[3.51-PEG(2)

Poly(L-lysine)hydrobromide (“PLL-HBr”) is dissolved in 25 ml sodium tetraborate buffer (“STBB”, 50 mM, pH 8.5) per gram PLL-HBr. The solution is stirred, then filtered (0.22 μm Durapore membrane, sterile Millex GV, Sigma-Aldrich, Buchs, Switzerland) and filled into a sterile culture tube. While the solution is constantly stirred, a suitable quantity of MeO-PEG-SPA powder is then added according to stoichiometric conditions. After a further six hours of stirring, the solution is transferred at room temperature to a dialysis tube (Spectr/Por dialysis tubes, molecular weight cut-off 6-8 kDa, Sochochim, Lausanne, Switzerland). The dialysis is carried out for 24 hours in a liter of phosphate-buffered saline (“PBS”, 10 mM, pH 7.0), followed by a another 24 hours of further dialysis in a liter of deionized water. The product is then lyophilized for 48 hours at a temperature −50° C. and a pressure of 0.2 mbar.

3.2 Synthesis of Biotinylated PLL-g-PEG

Biotinylated PLL-g-PEG is synthesized in a manner similar to that described hereinbefore. A suitable quantity of biotin PEG-CO₂-NHS powder according to the stiochiometric conditions is slowly added to the filtered solution of PLL-HBr solution and stirred for one hour. A suitable quantity of MeO-PEG-SPA according to the stiochiometric conditions is then added, and the resulting solution is stirred for a further five hours. The further steps of dialysis and product extraction are identical to those described hereinbefore.

3.3 Determination of the grafting ratio and the percentage proportion of biotin The grafting ratio and the percentage proportion of biotin in the biotinylated PEG derivatives are estimated by means of 1H-NMR. The lyophilized polymers are dissolved in D₂O and the spectra recorded using a 300 MHz NMR spectrometer. The resulting values are presented in Table 1.

TABLE 1
Grafting ratio and percentage proportion of biotin in biotinylated
PEG derivatives, determined by means of 1H-NMR.

| Name of polymer | Grafting ratio | % biotinylated PEG chains |
|---|---|---|
| PLL-g-PEG | 4.4 +/− 1.5 | 0 |
| PPB20 | 3.2 +/− 1.0 | 23 +/− 7 |
| PPB30 | 3.9 +/− 0.5 | 31 +/− 5 |
| PPB50 | 2.8 +/− 0.5 | 51 +/− 5 |

4. Determination of the Quantity of Molecules Adsorbed to the Surface Using a Grating Coupler Sensor as Carrier

The mass of polymer adsorbed to the Nb₂O₅ surfaces is determined on the basis of the difference in coupling conditions for the in-coupling of light into a grating coupler sensor before and after application of the respective polymer layers. The working principle of a grating coupler sensor is described for example in U.S. Pat. No. 4,952,056. A grating coupler device (BIOS I, ASI AG, Zürich, Switzerland) was used as the measuring instrument.

The values of the mass of surface-adsorbed material are determined using the Feijter's formula (J. J. Ramsden, J. Stat. Phys. 73 (1993) 853-877). By means of a Raleigh interferometer, an incremental value of dn/dc=0.158 cm³/g was determined for the adsorption of the polymers on a surface and used as a basis for the further calculations. The protein adsorption on a surface was conditional upon a value of dn/dc=0.182 cm³/g (J. J. Ramsden, D. J. Roush, D. S. Gill, R. Kurrat, R. C. Willson, J. Am. Chem. Soc. 117 (1995) 8511-8516).

5. Coating of Nb₂O₅ Surfaces with Polymers

A carrier pretreated according to section 2. of this example is equilibrated in HEPES-1 buffer (10 mM HEPES, pH 7.4) for at least five hours before an experiment, then inserted into the grating coupler measuring instrument and equilibrated there for a further hour in HEPES-1 buffer, until a stable baseline, i.e. a stable resonance angle for in-coupling of the excitation light into the highly refractive waveguiding layer by means of the coupling grating, is achieved.

Solutions of PLL-g-PEG (15 μM) and PLL-g-PEG/PEG biotin in HEPES-1 buffer are filtered through 0.22 μm Durapore membranes and mixed immediately before use. The Nb₂O₅ surfaces are coated in situ in the grating coupler device by bringing the metal oxide surface of the carrier into contact with the solution of the polymer mixture. This is carried out over a period of 30 minutes under constant flow at a flow rate of 1 ml/h. The carrier thus coated is then rinsed for 30 minutes with HEPES-1 buffer.

6. Procedure of Protein Binding Assay

6.1. Standard Assay Protocol

For most of the experiments described hereinafter, the polymer-coated carriers are incubated sequentially under continuous flow (flow rate: 1 ml/h) with solutions of streptavidin (100 μg/ml), anti-R-IgG biotin (100 μg/ml) and finally R-IgG (200 μg/ml). Each of these incubation steps lasts 15 minutes, which—at the concentrations selected—is sufficient for saturation of all available binding sites. There then follows a 30-minute washing step with the respective buffer to remove nonbound molecules which had been added before. In each case, HEPES buffer solutions are used.

6.2. Special Assay Protocols

For studies described hereinafter with regard to the influence of streptavidin surface densities on binding behavior, the same procedural steps are used as described under 6. 1., except that a streptavidin concentration of only 2.5 μg/ml is used and the incubation time amounts to 45 minutes.

For studies on the dependence of the optimal surface density of recognition elements on the size of the molecules to be bound from an added sample, BSA biotin (100 μg/ml) and rabbit anti-bovine serum albumin (“anti-BSA”, 200 μg/ml) is used instead of anti-R-IgG biotin and R-IgG.

7. Application of Polymers with Partly Bound Recognition Elements on a Carrier

A thin-film waveguide formed as a grating coupler (TiO₂—SiO₂-solgel as waveguiding layer on a glass substrate, coupling grating period in the waveguiding layer: 417 nm), with a 12 nm thick Nb₂O₅ layer deposited thereon, serves as carrier. At an adjusted pH of 7.4 of a subsequently applied solution, the surface of Nb₂O₅ is negatively charged (isoelectric point IEP=3.6), whereas PLL main chains of PLL-g-PEG and PLL-g-PEG/PEG biotin are highly positively charged. It is assumed that the strong adsorption of polymer comprising PLL as major component on Nb₂O₅-coated surfaces is based in particular on electrostatic interaction between this metal oxide surface and the polymer as multiply charged adsorbate.

The aim of applying a mixture of PLL-g-PEG and PLL-g-PEG/PEG biotin is to achieve an optimal binding capacity of the polymer-coated surface by adjusting the ratio of the mixture and at the same time to minimize nonspecific binding. Biotin, bound as a recognition element in the polymers PLL-g-PEG/PEG biotin, serves as a specific recognition element for molecules such as avidin or streptavidin, to which in a further binding step “biotinylated” molecules (i.e. molecules associated with biotin), such as anti-R-IgG biotin, can be bound, and which in turn can serve as recognition elements for an analyte (in this example R-IgG).

As a result of the application of this polymer mixture with PLL-g-PEG/PEG biotin on the Nb₂O₅ surface, the biotin binding sites are surrounded by nonbinding PEG chains, so that protein adsorption is reduced. Such properties are described in a series of patents (e.g. in U.S. Pat. Nos. 5,820,882, 5,232,984, 5,380,536, 6,231,892, 5,462,990, 5,627,233, and 5,849,839). In WO 00/65352, polymers of this kind are also described with biotin molecules bound thereto. In these prior art documents, however, there is no reference to the fact that—as according to the present invention—the binding capacity can be optimized by adjusting the proportion of bound biotin. This optimization of the binding capacity is achieved according to the invention, in this example by an adjustment of the proportions of PLL-g-PEG and PLL-g-PEG/PEG biotin.

The mass of polymer adsorbed to the Nb₂O₅ surfaces is determined on the basis of the difference in coupling conditions for the in-coupling of light into a grating coupler before and after deposition of the respective polymer layers. This results in values of 167±8 ng/cm² adsorbed polymer being determined for pure PLL-g-PEG and 213±13 ng/cm for pure PPB20. Taking into account the molecular weights and the grating ratio determined by means of NMR, the surface concentrations of adsorbed polymers are determined for every mixture ratio used. Within the experimental accuracy, a uniform value of 2.5±0.1 pmol/cm² is obtained, from which it is concluded that the mixture ratio of the polymers on the surface is the same as before in solution.

8. Binding of Streptavidin to the Recognition Surface

Upon application of a solution of streptavidin (100 μg/ml) to surfaces with different mixture ratios (PLL-g-PEG: PPB20), under continuous flow, a saturation signal for the binding of streptavidin to the surface coated with biotinylated polymers is attained within 15 minutes. After a washing step with HEPES-1 buffer, the quantity of bound streptavidin is determined in each case for the surfaces with different polymer mixtures. The quantity of surface-bound biotin is determined from NMR measurements and from the mixture ratio of the polymers in solution.

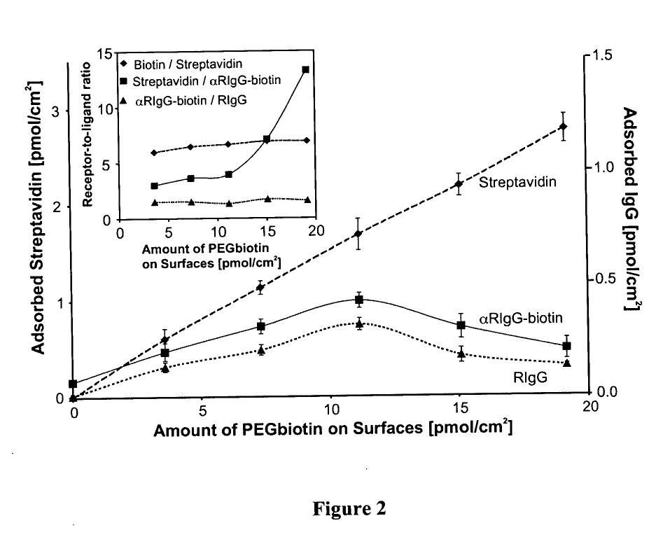

In this series of measurements with different mixture ratios of surface-immobilized polymers, the proportion of bound streptavidin continuously rises with the proportion of PPB20 (see FIG. 2). No binding or adsorption is observed on the pure PLL-g-PEG surface, whereas 2.77 pmol/cm² streptavidin is bound to a pure PPB20 surface (corresponding to 19.2 pmol/cm² biotin). From the two-dimensional crystal structure of streptavidin, it can be concluded that an individual streptavidin molecule is about 5.5 nm×4.5 nm in size (S. A. Darst, M. Ahlers, P. H. Meller, E. W. Kubalek, R. Blankenburg, H. O. Ribi, H. Ringsdorf, R. D. Komberg, Biophys. J. 59 (1991) 387-396). Consequently, for a densely packed streptavidin monolayer, a surface density of 6.71 pmol/cm² can be expected. The surface densities of streptavidin which are bound to the surface under the conditions of the experiment leading to FIG. 2 thus correspond to between 0% and 41% of a densely packed monolayer.

For all polymer mixture ratios used in this series, the ratio of bound streptavidin to surface-immobilized biotin is 1:6.5. The relatively high surplus of biotin submolecules as immobilized recognition elements versus bound streptavidin which was applied in an excess that would inevitably lead in quantitative terms to the saturation of all available binding sites can be explained by the fact that some of the biotin molecules might not be accessible on the surface, but might be concealed in the PEG sublayer. On the other hand, binding of a streptavidin molecule to two or more biotin molecules might also have taken place.

In the case of a surface on which pure PPB50 is immobilized (corresponding to 42 pmol/cm² biotin on the surface) about 6.65 pmol/cm² streptavidin is bound, which corresponds approximately to the quantity of a monolayer. In this case, the ratio of bound streptavidin to immobilized biotin is thus about 1:5.

In the next step, biotinylated anti-rabbit immunoglobulin (anti-R-IgG biotin) is bound to the surface modified beforehand with streptavidin (to the remaining binding sites for biotin to streptavidin). There then follows a washing step with HEPES-1 buffer.

FIG. 2 shows the quantity of bound anti-R-IgG biotin as a function of the concentration of initially surface-bound biotin. With increasing biotin surface concentration, i.e. with a simultaneously increasing concentration or surface density of bound streptavidin, the quantity of bound anti-R-IgG biotin also increases at first. With a surface concentration (density) of about 11.2 pmol/cm² of biotin bound via PEG/biotin, corresponding to a concentration of 1.68 pmol/cm² of bound streptavidin (or x % of a complete monolayer), a maximum of about 0.43 pmol/cm²,of the quantity of bound anti-R-IgG biotin, is reached. With a further increase in the density of surface-immobilized PEG-biotin and thus of the streptavidin bound thereto, the quantity of bound anti-R-IgG biotin falls again.

The decrease in the binding capacity for anti-R-IgG biotin may be explained by steric hindrance of the sites available for binding to streptavidin. It also has to be taken into account here that the anti-R-IgG biotin molecule with a size similar to that of anti-R-IgG (namely 14.3 nm×5.9 nm×13.1 nm (H. D. Kratzin, W. Plam, M. Stangel, W. E. Schmidt, J. Friedrich, N. Hilschmann, Biol. Chem. H-S 370 (1989) 263-272)) covers an area approximately 2.5 times larger than that of streptavidin if one assumes a footprint measuring 14.3 nm×5.9 nm.

To test the hypothesis of a decrease in binding capacity due to steric hindrance at a high density of relatively large surface-bound recognition elements, a further experiment is carried out in which, instead of anti-R-IgG biotin (molecular weight about 150,000), the smaller protein BSA biotin (molecular weight about 50,000) is added, followed in the next step by addition of the antibody anti-BSA.

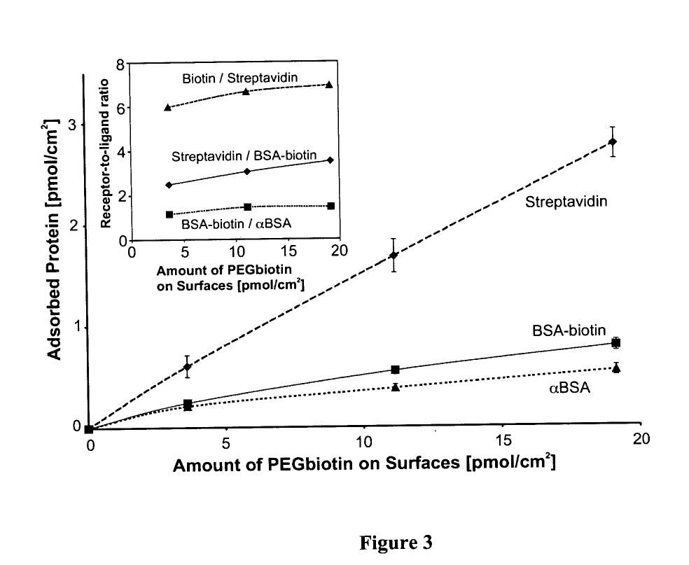

FIG. 3 shows the results for the sequential adsorption or binding of streptavidin, BSA biotin and anti-BSA to the carrier surface coated with mixed polymer layers: The quantity of bound BSA biotin continuously increases with the quantity of surface-bound biotin, rising well beyond the value at which the maximum was achieved for the streptavidin/anti-E-IgG biotin system. Up to surface concentrations of 2.77 pmol/cm² streptavidin, no maximum value of bound BSA biotin (about 0.7 pmol/cm² at 2.77 pmol/cm² streptavidin or 11.6 pmol/cm² surface-bound PEG/biotin) is attained.

In a further experiment, pure PPB50 is immobilized on the carrier surface. In the further corresponding assay steps, 6.65 pmol/cm² streptavidin binds to the surface, compared with only 0.12 pmol/cm² BSA biotin, from which it can be concluded that, for this smaller molecule, the immobilization density of the recognition elements likewise has an optimum value, which is shifted however to higher values of surface-bound streptavidin (between 2.77 pmol/cm² and 6.65 pmol/cm²).

In a last assay step, the “chip” which was brought into contact beforehand with anti-R-IgG biotin is brought into contact with R-IgG as analyte and then rinsed with buffer. As shown in FIG. 4, the binding behavior for R-IgG very closely follows the trend of the binding curve of anti-R-IgG biotin, as described hereinbefore for the binding of anti-R-IgG biotin.

1. A recognition surface with an (in relation to the surface) optimal binding capacity for recognizing and binding one or more analytes from one or more samples brought into contact with this surface, wherein
a) said recognition surface comprises a mixture of specific biological or biochemical or synthetic recognition elements for the recognition and binding of said analytes with components which are “neutral” in respect of these analytes, i.e. which do not bind these analytes, and
b) said specific recognition elements, in relation to the entire recognition surface or any sub-surface thereof, take up less than a full monolayer.

2. A recognition surface according to claim 1, wherein said specific recognition elements, in relation to the entire recognition surface or any sub-surface thereof, form one-tenth to half of a complete monolayer.

3. A recognition surface according to claim 1, wherein said specific recognition elements and components “neutral” to the analytes, in relation to the entire recognition surface or any sub-surface thereof, together form at least two-thirds of a complete monolayer.

4. A structured recognition surface with an (in relation to the surface) optimal binding capacity for recognizing and binding one or more analytes from one or more samples brought into contact with this surface, wherein
a) said recognition surface in discrete, laterally separated measurement areas comprises a mixture of specific biological or biochemical or synthetic recognition elements for the recognition and binding of said analytes with components which are “neutral” in respect of these analytes, i.e. which do not bind these analytes, and
b) said specific recognition elements, in relation to the surface area of the discrete measurement areas, take up less than a full monolayer.

5. A structured recognition surface according to claim 4, wherein said specific recognition elements, in relation to the entire recognition surface or any sub-surface thereof, form one-tenth to half of a complete monolayer.

6. A structured recognition surface according to claim 4 wherein said specific recognition elements and components “neutral” to the analytes, in relation to the entire recognition surface or any sub-surface thereof, together form at least two-thirds of a complete monolayer.

7. A structured recognition surface according to claim 4, wherein up to 1,000,000 measurement areas are provided in a 2-dimensional arrangement and a single measurement area covers an area of 10⁻⁴ mm²-10 mm².

8. A structured recognition surface according to claim 4, wherein the measurement areas are arranged in a density of more than 10, preferably more than 100, especially preferably more than 1000 measurement areas per square centimeter.

9. A structured recognition surface according to claim 4, wherein discrete (laterally separated) measurement areas, as an integral part of this recognition surface, are generated by the laterally selective application of biological or biochemical or synthetic recognition elements on a surface of a carrier or on an adhesion-promoting layer additionally applied to a carrier surface, preferably using one or more methods from the group of methods comprising ink-jet spotting, mechanical spotting by means of pin, pen or capillary, micro-contact printing, fluidic contact of the measurement areas with the biological or biochemical or synthetic recognition elements through their application in parallel or intersecting microchannels, upon exposure to pressure differences or to electric or electromagnetic potentials, and photochemical or photolithographic immobilization methods.

10. A structured recognition surface according to claim 4, wherein regions between the laterally separated measurement areas are “passivated” in order to minimize nonspecific binding of analytes or their tracer compounds, i.e. that compounds are deposited between the laterally separated measurement areas which are “chemically neutral” to the analyte or one of its tracer compounds, formed preferably for example from groups comprising albumins, especially bovine serum albumin or human serum albumin, casein, nonspecific polyclonal or monoclonal, heterologous or empirically nonspecific antibodies for the analyte or analytes to be determined (especially for immunoassays), detergents (such as Tween 20®), fragmented natural or synthetic DNA not hybridizing with polynucleotides to be analyzed, such as extract from herring or salmon sperm (especially for polynucleotide hybridization assays), or also uncharged but hydrophilic polymers, such as polyethylene glycols or dextrans.

11. A recognition surface according to claim 1, wherein said biological or biochemical or synthetic recognition elements are selected from the group formed from proteins, for example monoclonal or polyclonal antibodies and antibody fragments, peptides, enzymes, aptamers, synthetic peptide structures, glycopeptides, oligosaccharides, lectins, antigens for antibodies (e.g. biotin for streptavidin), proteins functionalized with additional binding sites (“tag proteins”, such as “histidine tag proteins”) and their complexing partners.

12. A recognition surface according to claim 1, wherein said biological or biochemical or synthetic recognition elements are selected from the group comprising nucleic acids (for example DNA, RNA, oligonucleotides) and nucleic acid analogs (e.g. PNA) as well as their derivatives with synthetic bases.

13. A recognition surface according to claim 1, wherein said biological or biochemical or synthetic recognition elements are selected from the group comprising soluble, membrane-bound proteins and proteins isolated from a membrane, such as receptors and their ligands.

14. A recognition surface according to claim 1, wherein said “neutral” components which do not bind the analyte or analytes may be selected from groups comprising albumins, especially bovine serum albumin or human serum albumin, casein, nonspecific, polyclonal or monoclonal, heterologous or for the analyte or analytes to be determined empirically nonspecific antibodies (especially for immunoassays), detergents (such as Tween 20), fragmented natural or synthetic DNA not hybridizing with polynucleotides for analysis, such as a herring or salmon sperm extract (especially for polynucleotide hybridization assays), or also uncharged, but hydrophilic polymers, such as polyethylene glycols or dextrans.

15. A recognition surface according to claim 1, wherein said recognition elements are bound to the free end or close to the free end of a wholly or partly functionalized, “noninteractive” polymer, wherein said “noninteractive” polymer as a side chain is bound to a charged, polyionic polymer as the main chain and, together with this polymer, forms a polyionic, multifunctional copolymer.

16. A recognition surface according to claim 15, wherein the polyionic polymer main chain is cationically (positively) charged at approximately neutral pH.

17. A recognition surface according to claim 16, wherein the cationic main chain is selected from the group of polymers comprising amino acids with a positive charge at approximately neutral pH, polysaccharides, polyamines, polymers of quartemary amines and charged synthetic polymers.

18. A recognition surface according to claim 17, wherein the cationic polymer main chain comprises one or more molecular groups from the group comprising lysine, histidine, arginine, chitosan, partially deacetylated chitin, amine-containing derivatives of neutral polysaccharides, polyaminostyrene, polyamine acrylates, polyamine methacrylates, polyethylene imines, polyamine ethylenes, polyaminostyrenes and N-alkyl derivatives thereof.

19. A recognition surface according to claim 15, wherein the polyionic polymer main chain is anionically (negatively) charged at approximately neutral pH.

20. A recognition surface according to claim 19, wherein the cationic main chain is selected from the group of polymers comprising amino acids with associated groups having a negative charge at approximately neutral pH, polysaccharides and charged synthetic polymers with negatively charged groups.

21. A recognition surface according to claim 20, wherein the cationic polymer main chain comprises one or more groups of molecules from the group comprising polyasparaginic acid, polyglutamic acid, alginic acid or derivatives thereof, pectin, hyaluronic acid, heparin, heparin sulfate, chondroitin sulfate, dermatan sulfate, dextran sulfate, polymethyl methacrylic acid, oxidized cellulose, carboxymethylated cellulose, maleic acid and fumaric acid.

22. A recognition surface according to claim 15, wherein the “noninteractive” polymer” as side chain is selected from the group comprising poly(alkylene glycols), poly(alkylene oxides), neutral water-soluble polysaccharides, polyvinyl alcohols, poly-N-vinyl pyrrolidones, phosphorylcholine derivatives, noncationic poly(meth)acrylates and combinations thereof.

23. A recognition surface according to claim 15, wherein the biological or biochemical or synthetic recognition elements are bound to the free end or close to the free end of the “noninteractive” side chain via reactive groups:

24. A recognition surface according to claim 23, wherein said reactive groups are selected from the group comprising hydroxy (—OH), carboxy (—COOH), esters (—COOR), thiols (—SH), N-hydroxysuccinimide, maleimidyl, quinone, vinylsulfone, nitrilo triacetic acid (NTA) and combinations thereof.

25. A recognition surface according to claim 1, wherein this is deposited on an essentially optically transparent carrier.

26. A recognition surface according to claim 25, wherein the essentially optically transparent carrier comprises a material from the group comprising moldable, sprayable or millable plastics, metals, metal oxides, silicates, such as glass, quartz or ceramics.

27. A recognition surface according to claim 25, wherein the recognition surface is deposited on an adhesion-promoting layer which is applied to an essentially optically transparent carrier and which is likewise essentially optically transparent.

28. A recognition surface according to claim 27, wherein the adhesion-promoting layer is less than 200 nm thick, and preferably less than 20 nm.

29. A recognition surface according to claim 27, wherein the adhesion-promoting layer comprises a chemical compound from the group of silanes, functionalized silanes, epoxides, functionalized, charged or polar polymers and “self-assembled passive or functionalized monolayers or multilayers”.

30. A recognition surface according to claim 25, wherein recesses are formed in the surface of said carrier for the creation of sample compartments.

31. A recognition surface according to claim 30, wherein said recesses have a depth of 20 μm to 500 μm, especially preferably 50 μm to 300 μm.

32. A recognition surface according to claim 25, wherein the essentially optically transparent carrier comprises a continuous optical waveguide or an optical waveguide divided into individual waveguiding areas.

33. A recognition surface according to claim 32, wherein the optical waveguide is an optical film waveguide with a first essentially optically transparent layer (a) facing the recognition surface on a second essentially optically transparent layer (b) with a refractive index lower than that of layer (a).

34. A recognition surface according to claim 33, wherein said optical film waveguide is essentially planar.

35. A recognition surface according to claim 32, wherein, for the in-coupling of excitation light into the optically transparent layer (a), this layer is in optical contact with one or more optical in-coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

36. A recognition surface according to claim 35, wherein the excitation light is in-coupled into the optically transparent layer (a) using one or more grating structures (c) which are featured in the optically transparent layer (a).

37. A recognition surface according to claim 35, wherein light guided in the optically transparent layer (a) is out-coupled using one or more grating structures (c′) which are featured in the optically transparent layer (a) and have the same or different period and grating depth as grating structures (c).

38. A method for the qualitative and/or quantitative detection of one or more analytes in one or more samples, wherein said samples and if necessary further reagents are brought into contact with a recognition surface according to claim 1, and changes in optical or electronic signals resulting from the binding of the analyte or of further tracer substances used for the analyte detection are measured.

39. A method for the qualitative and/or quantitative detection of one or more analytes in one or more samples, wherein said samples and if necessary further reagents are brought into contact with a structured recognition surface according to claim 4, and changes in optical or electronic signals emanating from the discrete measurement areas as a result of the binding of the analyte or of further tracer substances used for the analyte detection are measured in a locally resolved manner.

40. A method according to claim 38, wherein one or more samples are pre-incubated with a mixture of the various tracer reagents for determining the analytes to be detected in said samples, and these mixtures are then brought into contact with said recognition surface in a single addition step.

41. A method according to claim 38, wherein the detection of one or more analytes is based on the determination of the change in one or more luminescences.

42. A method according to claim 38, wherein the excitation light from one or more light sources is delivered in an epi-illumination configuration.

43. A method according to claim 38, wherein the excitation light from one or more light sources is delivered in a transillumination configuration.

44. A method according to claim 38, wherein the recognition surface, mediated if necessary by means of an adhesion-promoting layer, is arranged on an optical waveguide, which is preferably essentially planar, wherein one or more samples with one or more analytes to be detected therein and, if necessary further tracer reagents, are brought sequentially or in a single addition step after mixture with said samples, into contact with said recognition surface, and wherein the excitation light from one or more light sources is in-coupled into the optical waveguide using one or more optical coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

45. A method according to claim 44, wherein the detection of one or more analytes on a recognition surface takes place via a grating structure (c) or (c′) formed in the layer (a) of an optical film waveguide based on changes in the resonance conditions for the in-coupling of excitation light into layer (a) of a carrier formed as film waveguide or for out-coupling of light guided in layer (a), these changes resulting from binding of the analyte and/or further tracer reagents to their immobilized biological or biochemical or synthetic recognition elements.

46. A method according to claim 44, wherein said optical waveguide is designed as an optical film waveguide with a first optically transparent layer (a) on a second optically transparent layer (b) with lower refractive index than layer (a), wherein excitation light is further in-coupled into the optically transparent layer (a) with the aid of one or more grating structures, which are featured in the optically transparent layer (a), and delivered as a guided wave to measurement areas (d) located thereon, and wherein the luminescence of molecules capable of luminescence, generated in the evanescent field of said guided wave, is further determined using one or more detectors, and the concentration of one or more analytes is determined from the intensity of these luminescence signals.

47. A method according to claim 44, wherein (1) the isotropically emitted luminescence or (2) luminescence in-coupled into the optically transparent layer (a) and out-coupled via grating structure (c) or (c′) or luminescences of both (1) and (2) are measured simultaneously.

48. A method according to claim 46, wherein, for the generation of luminescence, a luminescence dye or luminescent nanoparticle is used as a luminescence label, which can be excited and emits at a wavelength between 300 nm and 1100 nm.

49. A method according to claim 48, wherein the luminescence label is bound to the analyte or, in a competitive assay, to an analog of the analyte or, in a multistep assay, to one of the binding partners of the immobilized biological or biochemical or synthetic recognition elements or to the biological or biochemical or synthetic recognition elements.

50. A method according to claim 48, wherein a second luminescence label or further luminescence labels are used with excitation wavelengths either the same as or different from that of the first luminescence label and the same or different emission wavelength.

51. A method according to claim 50, wherein the second or further luminescence labels can be excited at the same wavelength as the first luminescence dye, but emit at different wavelengths.

52. A method according to claim 51, wherein the excitation spectra and emission spectra of the luminescence dyes used overlap only little or not at all.

53. A method according to claim 52, wherein charge or optical energy transfer from a first luminescence dye serving as donor to a second luminescence dye serving as acceptor is used for the purpose of detecting the analyte.

54. A method according to claim 46, wherein changes in the effective refractive index on the measurement areas are determined in addition to the determination of one or more luminescences.

55. A method according to claim 46, wherein the one or more luminescences and/or determinations of light signals at the excitation wavelength are carried out using a polarization-selective procedure.

56. A method according to claim 46, wherein the one or more luminescences are measured at a polarization different from that of the excitation light.

57. A method according to claim 38 for simultaneous or sequential, quantitative or qualitative determination of one or more analytes from the group of proteins, such as antibodies or antigens, receptors or ligands, chelators, functionalized proteins with one or more additional binding sites (“tag proteins” such as “histidine tag proteins”) and complexing partners thereof, oligonucleotides, DNA or RNA strands, DNA or RNA analogs, enzymes, enzyme cofactors or inhibitors, lectins and carbohydrates.

58. A method according to claim 38, wherein the samples to be analyzed are aqueous solutions, especially buffer solutions or naturally occurring body fluids such as blood, serum, plasma, lymph or urine or tissue fluids or yolk.

59. A method according to claim 38, wherein the sample to be analyzed is an optically turbid fluid, surface water, a soil or plant extract, a biological or synthetic process broth.

60. A method according to claim 38, wherein the samples to be analyzed are prepared from biological tissue parts or cell cultures.

61. Use of a recognition surface according to claim 1 for quantitative or qualitative analyses for the determination of chemical, biochemical or biological analytes in screening methods in pharmaceutical research, combinatorial chemistry, clinical and pre-clinical development, for real-time binding studies and the determination of kinetic parameters in affinity screening and in research, for qualitative and quantitative analyte determinations, especially for DNA and RNA analytics and for the determination of genomic or proteomic differences in the genome, such as single nucleotide polymorphisms, for the measurement of protein-DNA interactions, for the determination of control mechanisms for mRNA expression and for protein (bio)synthesis, for the generation of toxicity studies and the determination of expression profiles, especially for the determination of biological and chemical marker compounds, such as mRNA, proteins, peptides or small-molecular organic (messenger) compounds, and for the determination of antibodies, antigens, pathogens or bacteria in pharmaceutical product research and development, human and veterinary diagnostics, agrochemical product research and development, for symptomatic and pre-symptomatic plant diagnostics, for patient stratification in pharmaceutical product development and for therapeutic drug selection, for the determination of pathogens, nocuous agents and germs, especially of salmonella, prions, viruses and bacteria, especially in food and environmental analytics.

62. A method for the qualitative and/or quantitative detection of one or more analytes in one or more samples, wherein said samples and if necessary further reagents are brought into contact with a recognition surface according to claim 2, and changes in optical or electronic signals resulting from the binding of the analyte or of further tracer substances used for the analyte detection are measured.

63. A method for the qualitative and/or quantitative detection of one or more analytes in one or more samples, wherein said samples and if necessary further reagents are brought into contact with a recognition surface according to claim 3, and changes in optical or electronic signals resulting from the binding of the analyte or of further tracer substances used for the analyte detection are measured.

64. A method for the qualitative and/or quantitative detection of one or more analytes in one or more samples, wherein said samples and if necessary further reagents are brought into contact with a recognition surface according to claim 4, and changes in optical or electronic signals resulting from the binding of the analyte or of further tracer substances used for the analyte detection are measured.

65. A method according to claim 39, wherein one or more samples are pre-incubated with a mixture of the various tracer reagents for determining the analytes to be detected in said samples, and these mixtures are then brought into contact with said recognition surface in a single addition step.

66. A method according to claim 62, wherein one or more samples are pre-incubated with a mixture of the various tracer reagents for determining the analytes to be detected in said samples, and these mixtures are then brought into contact with said recognition surface in a single addition step.

67. A method according to claim 63, wherein one or more samples are pre-incubated with a mixture of the various tracer reagents for determining the analytes to be detected in said samples, and these mixtures are then brought into contact with said recognition surface in a single addition step.

68. A method according to claim 64, wherein one or more samples are pre-incubated with a mixture of the various tracer reagents for determining the analytes to be detected in said samples, and these mixtures are then brought into contact with said recognition surface in a single addition step.

69. A method according to claim 39, wherein the detection of one or more analytes is based on the determination of the change in one or more luminescences.

70. A method according to claim 39, wherein the excitation light from one or more light sources is delivered in an epi-illumination configuration.

71. A method according to claim 39, wherein the excitation light from one or more light sources is delivered in a transillumination configuration.

72. A method according to claim 39, wherein the recognition surface, mediated if necessary by means of an adhesion-promoting layer, is arranged on an optical waveguide, which is preferably essentially planar, wherein one or more samples with one or more analytes to be detected therein and, if necessary further tracer reagents, are brought sequentially or in a single addition step after mixture with said samples, into contact with said recognition surface, and wherein the excitation light from one or more light sources is in-coupled into the optical waveguide using one or more optical coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

73. A method according to claim 72, wherein the detection of one or more analytes on a recognition surface takes place via a grating structure (c) or (c′) formed in the layer (a) of an optical film waveguide based on changes in the resonance conditions for the in-coupling of excitation light into layer (a) of a carrier formed as film waveguide or for out-coupling of light guided in layer (a), these changes resulting from binding of the analyte and/or further tracer reagents to their immobilized biological or biochemical or synthetic recognition elements.

74. A method according to claim 72, wherein said optical waveguide is designed as an optical film waveguide with a first optically transparent layer (a) on a second optically transparent layer (b) with lower refractive index than layer (a), wherein excitation light is further in-coupled into the optically transparent layer (a) with the aid of one or more grating structures, which are featured in the optically transparent layer (a), and delivered as a guided wave to measurement areas (d) located thereon, and wherein the luminescence of molecules capable of luminescence, generated in the evanescent field of said guided wave, is further determined using one or more detectors, and the concentration of one or more analytes is determined from the intensity of these luminescence signals.

75. A method according to claim 72, wherein (1) the isotropically emitted luminescence or (2) luminescence in-coupled into the optically transparent layer (a) and out-coupled via grating structure (c) or (c′) or luminescences of both (1) and (2) are measured simultaneously.

76. A method according to claim 74, wherein, for the generation of luminescence, a luminescence dye or luminescent nanoparticle is used as a luminescence label, which can be excited and emits at a wavelength between 300 nm and 1100 nm.

77. A method according to claim 76, wherein the luminescence label is bound to the analyte or, in a competitive assay, to an analog of the analyte or, in a multistep assay, to one of the binding partners of the immobilized biological or biochemical or synthetic recognition elements or to the biological or biochemical or synthetic recognition elements.

78. A method according to claim 76, wherein a second luminescence label or further luminescence labels are used with excitation wavelengths either the same as or different from that of the first luminescence label and the same or different emission wavelength.

79. A method according to claim 47, wherein changes in the effective refractive index on the measurement areas are determined in addition to the determination of one or more luminescences.

80. A method according to claim 47, wherein the one or more luminescences and/or determinations of light signals at the excitation wavelength are carried out using a polarization-selective procedure.

81. A method according to claim 47, wherein the one or more luminescences are measured at a polarization different from that of the excitation light.

82. A method according to claim 39 for simultaneous or sequential, quantitative or qualitative determination of one or more analytes from the group of proteins, such as antibodies or antigens, receptors or ligands, chelators, functionalized proteins with one or more additional binding sites (“tag proteins” such as “histidine tag proteins”) and complexing partners thereof, oligonucleotides, DNA or RNA strands, DNA or RNA analogs, enzymes, enzyme cofactors or inhibitors, lectins and carbohydrates.

83. A method according to claim 39, wherein the samples to be analyzed are aqueous solutions, especially buffer solutions or naturally occurring body fluids such as blood, serum, plasma, lymph or urine or tissue fluids or yolk.

84. A method according to claim 39, wherein the sample to be analyzed is an optically turbid fluid, surface water, a soil or plant extract, a biological or synthetic process broth.

85. A method according to claim 39, wherein the samples to be analyzed are prepared from biological tissue parts or cell cultures.

86. Use of a recognition surface according to claim 4 for quantitative or qualitative analyses for the determination of chemical, biochemical or biological analytes in screening methods in pharmaceutical research, combinatorial chemistry, clinical and pre-clinical development, for real-time binding studies and the determination of kinetic parameters in affinity screening and in research, for qualitative and quantitative analyte determinations, especially for DNA and RNA analytics and for the determination of genomic or proteomic differences in the genome, such as single nucleotide polymorphisms, for the measurement of protein-DNA interactions, for the determination of control mechanisms for mRNA expression and for protein (bio)synthesis, for the generation of toxicity studies and the determination of expression profiles, especially for the determination of biological and chemical marker compounds, such as mRNA, proteins, peptides or small-molecular organic (messenger) compounds, and for the determination of antibodies, antigens, pathogens or bacteria in pharmaceutical product research and development, human and veterinary diagnostics, agrochemical product research and development, for symptomatic and pre-symptomatic plant diagnostics, for patient stratification in pharmaceutical product development and for therapeutic drug selection, for the determination of pathogens, nocuous agents and germs, especially of salmonella, prions, viruses and bacteria, especially in food and environmental analytics.

87. Use of a method according to claim 38 for quantitative or qualitative analyses for the determination of chemical, biochemical or biological analytes in screening methods in pharmaceutical research, combinatorial chemistry, clinical and pre-clinical development, for real-time binding studies and the determination of kinetic parameters in affinity screening and in research, for qualitative and quantitative analyte determinations, especially for DNA and RNA analytics and for the determination of genomic or proteomic differences in the genome, such as single nucleotide polymorphisms, for the measurement of protein-DNA interactions, for the determination of control mechanisms for mRNA expression and for protein (bio)synthesis, for the generation of toxicity studies and the determination of expression profiles, especially for the determination of biological and chemical marker compounds, such as mRNA, proteins, peptides or small-molecular organic (messenger) compounds, and for the determination of antibodies, antigens, pathogens or bacteria in pharmaceutical product research and development, human and veterinary diagnostics, agrochemical product research and development, for symptomatic and pre-symptomatic plant diagnostics, for patient stratification in pharmaceutical product development and for therapeutic drug selection, for the determination of pathogens, nocuous agents and germs, especially of salmonella, prions, viruses and bacteria, especially in food and environmental analytics.

88. Use of a method according to claim 39 for quantitative or qualitative analyses for the determination of chemical, biochemical or biological analytes in screening methods in pharmaceutical research, combinatorial chemistry, clinical and pre-clinical development, for real-time binding studies and the determination of kinetic parameters in affinity screening and in research, for qualitative and quantitative analyte determinations, especially for DNA and RNA analytics and for the determination of genomic or proteomic differences in the genome, such as single nucleotide polymorphisms, for the measurement of protein-DNA interactions, for the determination of control mechanisms for mRNA expression and for protein (bio)synthesis, for the generation of toxicity studies and the determination of expression profiles, especially for the determination of biological and chemical marker compounds, such as mRNA, proteins, peptides or small-molecular organic (messenger) compounds, and for the determination of antibodies, antigens, pathogens or bacteria in pharmaceutical product research and development, human and veterinary diagnostics, agrochemical product research and development, for symptomatic and pre-symptomatic plant diagnostics, for patient stratification in pharmaceutical product development and for therapeutic drug selection, for the determination of pathogens, nocuous agents and germs, especially of salmonella, prions, viruses and bacteria, especially in food and environmental analytics.

89. A recognition surface according to claim 4, wherein said biological or biochemical or synthetic recognition elements are selected from the group formed from proteins, for example monoclonal or polyclonal antibodies and antibody fragments, peptides, enzymes, aptamers, synthetic peptide structures, glycopeptides, oligosaccharides, lectins, antigens for antibodies (e.g. biotin for streptavidin), proteins functionalized with additional binding sites (“tag proteins”, such as “histidine tag proteins”) and their complexing partners.

90. A recognition surface according to claim 4, wherein said biological or biochemical or synthetic recognition elements are selected from the group comprising nucleic acids (for example DNA, RNA, oligonucleotides) and nucleic acid analogs (e.g. PNA) as well as their derivatives with synthetic bases.

91. A recognition surface according to claim 4, wherein said biological or biochemical or synthetic recognition elements are selected from the group comprising soluble, membrane-bound proteins and proteins isolated from a membrane, such as receptors and their ligands.

92. A recognition surface according to claim 4, wherein said “neutral” components which do not bind the analyte or analytes may be selected from groups comprising albumins, especially bovine serum albumin or human serum albumin, casein, nonspecific, polyclonal or monoclonal, heterologous or for the analyte or analytes to be determined empirically nonspecific antibodies (especially for immunoassays), detergents (such as Tween 20), fragmented natural or synthetic DNA not hybridizing with polynucleotides for analysis, such as a herring or salmon sperm extract (especially for polynucleotide hybridization assays), or also uncharged, but hydrophilic polymers, such as polyethylene glycols or dextrans.

93. A recognition surface according to claim 4, wherein said recognition elements are bound to the free end or close to the free end of a wholly or partly functionalized, “noninteractive” polymer, wherein said “noninteractive” polymer as a side chain is bound to a charged, polyionic polymer as the main chain and, together with this polymer, forms a polyionic, multifunctional copolymer.

94. A recognition surface according to claim 93, wherein the polyionic polymer main chain is cationically (positively) charged at approximately neutral pH.

95. A recognition surface according to claim 94, wherein the cationic main chain is selected from the group of polymers comprising amino acids with a positive charge at approximately neutral pH, polysaccharides, polyamines, polymers of quarternary amines and. charged synthetic polymers.

96. A recognition surface according to claim 95, wherein the cationic polymer main chain comprises one or more molecular groups from the group comprising lysine, histidine, arginine, chitosan, partially deacetylated chitin, amine-containing derivatives of neutral polysaccharides, polyaminostyrene, polyamine acrylates, polyamine methacrylates, polyethylene imines, polyamine ethylenes, polyaminostyrenes and N-alkyl derivatives thereof.

97. A recognition surface according to claim 93, wherein the polyionic polymer main chain is anionically (negatively) charged at approximately neutral pH.

98. A recognition surface according to claim 97, wherein the cationic main chain is selected from the group of polymers comprising amino acids with associated groups having a negative charge at approximately neutral pH, polysaccharides and charged synthetic polymers with negatively charged groups.

99. A recognition surface according to claim 98, wherein the cationic polymer main chain comprises one or more groups of molecules from the group comprising polyasparaginic acid, polyglutamic acid, alginic acid or derivatives thereof, pectin, hyaluronic acid, heparin, heparin sulfate, chondroitin sulfate, dermatan sulfate, dextran sulfate, polymethyl methacrylic acid, oxidized cellulose, carboxymethylated cellulose, maleic acid and fumaric acid.

100. A recognition surface according to claim 93, wherein the “noninteractive” polymer” as side chain is selected from the group comprising poly(alkylene glycols), poly(alkylene oxides), neutral water-soluble polysaccharides, polyvinyl alcohols, poly-N-vinyl pyrrolidones, phosphorylcholine derivatives, noncationic poly(meth)acrylates and combinations thereof.

101. A recognition surface according to claim 93, wherein the biological or biochemical or synthetic recognition elements are bound to the free end or close to the free end of the “noninteractive” side chain via reactive groups:

102. A recognition surface according to claim 101, wherein said reactive groups are selected from the group comprising hydroxy (—OH), carboxy (—COOH), esters (—COOR), thiols (—SH), N-hydroxysuccinimide, maleimidyl, quinone, vinylsulfone, nitrilo triacetic acid (NTA) and combinations thereof.

103. A recognition surface according to claim 4, wherein this is deposited on an essentially optically transparent carrier.

104. A recognition surface according to claim 103, wherein the essentially optically transparent carrier comprises a material from the group comprising moldable, sprayable or millable plastics, metals, metal oxides, silicates, such as glass, quartz or ceramics.

105. A recognition surface according to claim 103, wherein the recognition surface is deposited on an adhesion-promoting layer which is applied to an essentially optically transparent carrier and which is likewise essentially optically transparent.

106. A recognition surface according to claim 105, wherein the adhesion-promoting layer is less than 200 nm thick, and preferably less than 20 nm.

107. A recognition surface according to claim 105, wherein the adhesion-promoting layer comprises a chemical compound from the group of silanes, functionalized silanes, epoxides, functionalized, charged or polar polymers and “self-assembled passive or functionalized monolayers or multilayers”.

108. A recognition surface according to claim 103, wherein recesses are formed in the surface of said carrier for the creation of sample compartments.

109. A recognition surface according to claim 108, wherein said recesses have a depth of 20 μm to 500 μm, especially preferably 50 μm to 300 μm.

110. A recognition surface according to claim 103, wherein the essentially optically transparent carrier comprises a continuous optical waveguide or an optical waveguide divided into individual waveguiding areas.

111. A recognition surface according to claim 110, wherein the optical waveguide is an optical film waveguide with a first essentially optically transparent layer (a) facing the recognition surface on a second essentially optically transparent layer (b) with a refractive index lower than that of layer (a).

112. A recognition surface according to claim 111, wherein said optical film waveguide is essentially planar.

113. A recognition surface according to claim 110, wherein, for the in-coupling of excitation light into the optically transparent layer (a), this layer is in optical contact with one or more optical in-coupling elements from the group comprising prism couplers, evanescent couplers with combined optical waveguides with overlapping evanescent fields, butt-end couplers with focusing lenses, preferably cylinder lenses, arranged in front of one face of the waveguiding layer, and grating couplers.

114. A recognition surface according to claim 113, wherein the excitation light is in-coupled into the optically transparent layer (a) using one or more grating structures (c) which are featured in the optically transparent layer (a).

115. A recognition surface according to claim 113, wherein light guided in the optically transparent layer (a) is out-coupled using one or more grating structures (c′) which are featured in the optically transparent layer (a) and have the same or different period and grating depth as grating structures (c).
